(19) United States
(12) Patent Application Publication
(10) Pub. No.: US 2005/0164371 A1
(43) Pub. Date: Jul. 28, 2005

(54) Cavity electrode structure, and sensor and protein detection device using the same

(75) Inventor: Kenji Arinaga, Kawasaki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: FUJITSU LIMITED, Kawasaki (JP)

(21) Appl. No.: 11/074,865

(22) Filed: Mar. 9, 2005

Related U.S. Application Data
(63) Continuation of application No. PCT/JP03/04030, filed on Mar. 28, 2003.

(51) Int. Cl. C12M 1/34

(52) U.S. Cl. 435/287.1

(57) ABSTRACT

A cavity electrode structure, which is provided with a pair of opposing electrodes having a precisely formed narrow gap, and a sensor and a protein detection device, in which the cavity electrode structure is used, are provided. The cavity electrode structure comprises a first electrode, an insulating layer located on this first electrode and having a through hole that partially exposes the first electrode, and a second electrode opposed to the exposed surface of the first electrode by protruding towards the inside of the through hole of the insulating layer and provided with an opening that leads to the through hole of the insulating layer, the structure having a cavity that is formed by the exposed surface of the first electrode, the inner walls of the through hole of the insulating layer, and the surface of the second electrode that opposes the first electrode. The sensor comprises an electrically conductive bridging member of which one end is fixed to the exposed surface of the first electrode of the aforementioned cavity electrode structure, while the other end is fixed to the opposing surface of the second electrode, and which has a site that specifically binds to a target protein to be detected. The protein detection device uses a bridging member provided with a site that specifically binds to a target protein to be detected.

### CROSS-REFERENCE TO RELATED APPLICATION

This application is a continuation application and is based upon PCT/JP03/04030, filed on Mar. 28, 2003.

### TECHNICAL FIELD

The present invention relates to a cavity electrode structure provided with a pair of opposing electrodes having a narrow gap precisely formed without being restricted by the spatial resolution of a photolithography process, and a sensor and a protein detection device using the same.

### BACKGROUND ART

Following its inception in the 1990s, various countries have shared the task of attempting to decipher all human gene codes, the results of which yielded the announcement of the completion of a draft of the human genome in the summer of 2000. The functions that are involved with each of the locations of the deciphered human genome sequence information are predicted to be identified in the future as a result of continuing progress in the areas of functional genome science and structural genome science.

This human genome project has brought about a major paradigm change in science, technology and industries related to the life sciences. For example, diabetes has been classified based on the symptom of elevated blood glucose levels, and the cause of its onset has been classified as type I (unable to produce insulin in the body) or type II (unable to regulate the amount of insulin in the body) based on the degree of the ability of the patient to produce insulin in the body. The human genome project has provided all information on the amino acid sequence structures of proteins such as enzymes and receptors involved in the detection of blood glucose and the synthesis, degradation and other regulatory aspects of insulin, as well as the DNA sequences of genes involved in control of the levels of those proteins. The use of such information allows diabetes to be classified as a phenomenon in which the regulation of blood glucose is not carried out normally into subtypes depending on which of the respective proteins involved in the series of processing consisting of glucose detection, insulin synthesis, insulin degradation and so forth is not functioning properly, which ought to enable the providing of appropriate diagnosis and treatment. In the pharmaceutical industry in particular, genome drug development is aggressively being conducted to develop drugs to specific proteins based on the human genome sequence, and it is predicted that the time will come when the status of such series of functionally related proteins will be determined for the purpose of administering genome-developed drugs and alleviating and curing symptoms.

Technology that enables the levels of such series of functionally related proteins to be measured easily is still in the developmental stage in the form of proteome analysis technology. Although currently established measurement methods consist of combining two-dimensional electrophoresis and a mass analyzer, these require comparatively elaborate equipment. It will be necessary to develop new, simpler technology in order to determine patient symptoms in the clinical setting such as in a hospital laboratory or at the bedside.

So-called DNA chips attempt to quantify the amount of DNA in a sample to be measured that has bound to a complementary DNA chain arranged in the form of an array on the chip based on fluorescent intensity by introducing a fluorescent pigment when preliminarily amplifying the DNA in the sample using a polymerase chain reaction (PCR). In contrast, protein cannot be quantified by a method equivalent to amplification by the PCR reaction as in the case of DNA. In addition, in the case a protein is present in a sample as a mixture of numerous types of proteins, there was the problem of the uniform introduction of a fluorescent label being unable to be used due to differences in the reactivity between individual proteins and the pigment.

In recent years, attempts have been made to produce protein detection devices using semiconductor processing technology, and high molecular weight biomolecules such as DNA have been attempted to be used as detection elements. In these devices using high molecular weight biomolecules, protein detection and quantification are typically carried out by arranging the detection DNA so as to be immobilized between a pair of electrodes followed by measuring the change in current that flows through the DNA. However, it has been extremely difficult to precisely form a pair of electrodes having a gap of several nanometers to several tens of nanometers equivalent to the size of DNA even with the use of leading-edge semiconductor processing technology. In the technology disclosed in Japanese Unexamined Patent Publication No. 3-128449, for example, a biosensor is produced by adhering a pair of electrodes to the surface of the same substrate using a semiconductor photolithography process. In addition, in the technology disclosed in Japanese National Publication No. 2000-501503, a biosensor is produced by a complex process consisting of etching the surface of a substrate, adhering electrodes to the etched portion by a photolithography process and then laminating another substrate onto this substrate. There are many aspects of the structures of these sensors that are dependent on the spatial resolution of semiconductor photolithography processes of the prior art, and are therefore inadequate for handling biomolecules on the nanometer scale, while conversely the use of sophisticated devices results in exorbitant costs.

### DISCLOSURE OF THE INVENTION

An object of the present invention is to provide a cavity electrode structure provided with a pair of opposing electrodes having a narrow gap precisely formed without being restricted by the spatial resolution of a photolithography process.

Another object of the present invention is to provide a sensor and protein detection device that use this electrode structure.

A cavity electrode structure of the present invention comprises: a first electrode, an insulating layer located on this first electrode and having a through hole that partially exposes the first electrode, and a second electrode opposed to the exposed surface of the first electrode by protruding towards the inside of the through hole of the insulating layer and provided with an opening that leads to the through hole of the insulating layer; said cavity electrode structure having a cavity that is formed by the exposed surface of the first electrode, the inner walls of the through hole of the insulating layer, and the surface of the second electrode that opposes the first electrode. This cavity structure can be formed by thin layer formation technology, and is therefore suitable for handling high molecular weight biomolecules on the nanometer scale.

A sensor of the present invention comprises an electrically conductive bridging member of which one end is fixed to the exposed surface of the first electrode of the aforementioned cavity electrode structure, while the other end is fixed to the opposing surface of the second electrode, and which has a site that specifically binds to a target substance to be detected. When this sensor is placed in an atmosphere containing the target substance to be detected, the sensor is able to detect the target substance to be detected according to a change in the electrical conductivity of the bridging member that occurs as a result of the target substance to be detected binding to the aforementioned site.

A protein detection device of the present invention is equivalent to that which applies the aforementioned sensor to the detection of protein. More specifically, this protein detection device is a protein detection device comprising an electrically conductive bridging member of which one end is fixed to the exposed surface of the first electrode of the aforementioned electrode structure, while the other end is fixed to the opposing surface of the second electrode, and which has a site that specifically binds to a target protein to be detected. When this device is placed in an atmosphere containing the target protein to be detected, the device detects the target protein to be detected according to a change in the electrical conductivity of the bridging member that occurs as a result of the target protein to be detected binding to the aforementioned site.

### BRIEF DESCRIPTION OF THE DRAWINGS

FIG. 1 is a schematic drawing illustrating an electrode structure of the present invention.

FIG. 2 is a schematic drawing illustrating a protein detection device according to the present invention.

FIG. 3 is an explanatory drawing of protein detection by the protein detection device of FIG. 2.

FIGS. 4A to 4C are schematic drawings illustrating the production of the cavity of a protein detection device of the present invention.

FIGS. 5A and 5B are schematic drawings illustrating the immobilization of DNA of a bridging member inside the cavity of a protein detection device.

FIG. 6 is a schematic drawing of a protein detection device ready for detection.

FIG. 7 is a schematic drawing of a protein detection device during the detection process.

### BEST MODE FOR CARRYING OUT THE INVENTION

An electrode structure of the present invention comprises a laminate composed of a first electrode, an insulating layer located on this first electrode and having a through hole that partially exposes the first electrode, and a second electrode opposed to the exposed surface of the first electrode by protruding towards the inside of the through hole of the insulating layer and having an opening that leads to the through hole of the insulating layer. In this laminate, the interval between the exposed surface of the first electrode and the surface of the second electrode in opposition thereto is determined by the thickness of the insulating layer located between them. An electrode structure of the present invention has a cavity demarcated by the exposed surface of the first electrode, the inner walls of the through hole provided in the insulating layer, and the surface of the second electrode that opposes the first electrode.

An electrode structure of the present invention can be used in research for investigating the electrical characteristics of DNA (such as the presence of electrical conductivity or semiconductor-like characteristics), investigating whether or not the electrical characteristics change according to differences in the base sequence, or investigating the manner in which the electrical characteristics of DNA are affected by the surrounding environment, by arranging DNA between the opposing first and second electrodes that form a cavity by fixing, for example, the ends of the DNA to the respective electrodes. Alternatively, an electrode structure of the present invention can be also used in research that applies various substances to molecule elements by arranging a specific substance between the first and second electrodes and investigating its electrical characteristics. In addition, an electrode structure of the present invention can be applied to various research fields relating to the electrical characteristics of various substances at the molecular level.

The interval between the opposing electrodes in an electrode structure of the present invention is 100 nanometers or less. The reason for this is that the length of DNA molecules or molecules that compose molecular elements handled by an electrode structure of the present invention is normally from several nanometers to several tens of nanometers. The gap between the electrodes must be correspondingly narrow in order to immobilize such extremely short molecules between the opposing electrodes. The production of a pair of opposing electrodes having a narrow gap on the order of several nanometers to several tens of nanometers using photolithography used in semiconductor processes is extremely difficult and the costs are prohibitively high.

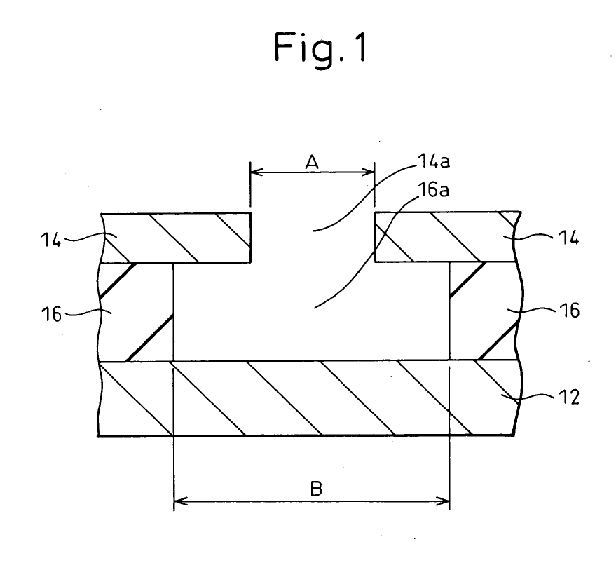

A schematic drawing of an electrode structure of the present invention is shown in FIG. 1. In electrode structure 10 of this drawing, the gap between first and second electrodes 12 and 14 is determined by the thickness of an insulating layer 16 interposed between them. Insulating layer 16 is formed using thin film formation technology, and the formation of a layer having a thickness on the order of several nanometers to several tens of nanometers is extremely easy using this technology. Moreover, the thickness of the insulating layer 16 formed can be changed as desired. Thus, an electrode structure of the present invention capable of being produced by thin film technology can be produced with much higher precision, better reproducibility and inexpensively in comparison with the use of an ordinary planar process or micromachine process dependent on photolithography. Although photolithography is used for forming opening 14a in upper electrode 14 and for forming through hole 16a in insulating layer 16, since the width or diameter A of opening 14a is typically required to be roughly 1 to 100 micrometers, while the width or diameter B of through hole 16a is typically required to be roughly 1 to 1000 micrometers, the processing accuracy required for their formation is not that severe in comparison with the accuracy of the thickness of the insulating layer that governs the gap between the electrodes. For this reason, photolithography is adequate for forming the through hole of an insulating film.

An electrode structure of the present invention can be used as a sensor by providing an electrically conductive member having a site (binding site) that specifically binds to a target substance to be detected and which connects both of the opposing electrodes of the electrode structure of the present invention by bridging the gap between them (in the present invention, this member is referred to as a “bridging member”) by having one end fixed to one of the electrodes and the other end fixed to the other electrode. A sensor of the present invention, when placed in an atmosphere containing a target substance to be detected, is able to detect the target substance to be detected according to a change in the electrical conductivity of the bridging member that occurs as a result of the target substance to be detected binding to the aforementioned site. The target substance to be detected may be present in a liquid phase or a gaseous phase. In other words, a sensor of the present invention can be used to detect a specific target substance to be detected present in a liquid phase or gaseous phase.

For example, in the case of attempting to detect a protein with a sensor of the present invention, the bridging member can be made with a high molecular weight biomolecule as represented by a polynucleotide, using an antibody, aptamer or low molecular weight organic compound (e.g., biotin) for the protein detector, and attaching the detector to an intermediate location of the high molecular weight biomolecule chain. The protein detector constitutes a site that specifically binds to a protein of the target substance to be detected. In addition, in the case of attempting to detect a nucleic acid with a sensor of the present invention, an oligonucleotide chain of, for example, 10 to 50 residues, having a complementary sequence to the target nucleic acid to be detected can be used for the bridging member. In this case, the oligonucleotide chain itself serves as the site that specifically binds with the target nucleic acid to be detected, and the nucleic acid can be detected through a change in the electrical characteristics of the bridging member that occurs as a result of DNA or RNA having a complementary sequence binding to the oligonucleotide chain bridging member.

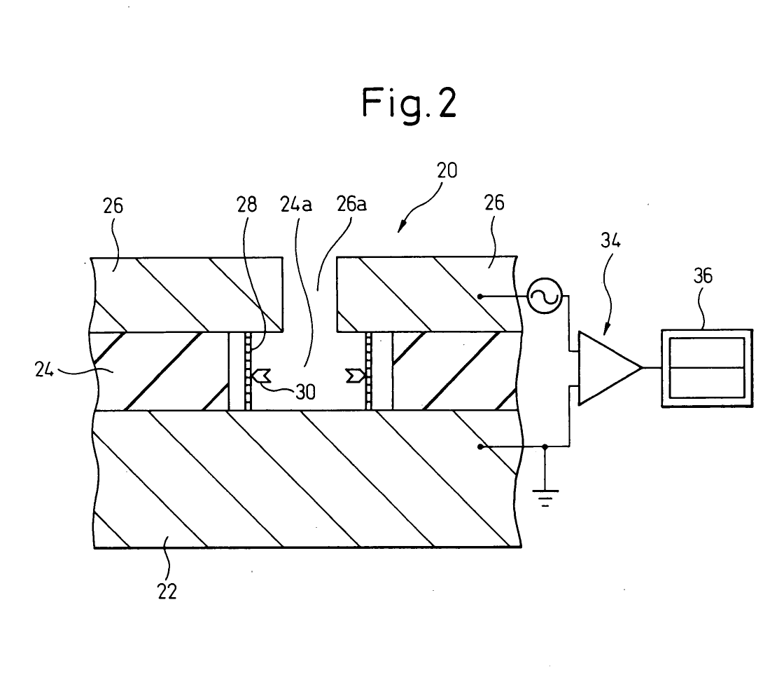

FIG. 2 shows a protein detection device 20 that applies a sensor of the present invention. An insulating layer 24 having a through hole 24a is positioned on lower electrode 22, and an upper electrode 26 having an opening 26a, which leads to through hole 24a and has a width or diameter smaller than the width or diameter of through hole 24a, is positioned thereon. A cavity is formed by the exposed surface (upper surface) of lower electrode 22, the inner walls of through hole 24g provided in insulating layer 24, and the surface of upper electrode 26 (lower surface) that opposes the exposed surface of lower electrode 22, and this cavity has a volume substantially equal to the internal volume of through hole 24a.

DNA 28 connects lower electrode 22 and upper electrode 26 by bridging the cavity in the form of a bridging member. A protein detector 30 that serves as the site that specifically binds to a target protein to be detected is attached to an intermediate location of DNA 28. Any substance such as antibody, aptamer or low molecular weight organic substance (e.g., biotin) that specifically binds to the target protein to be detected can be used for protein detector 30.

A protein detection device 20 shown in FIG. 2 is provided with a signal processing device 34 connected to lower electrode 22 and upper electrode 26 that processes signals (data) indicating a change in electrical characteristics that occurs as a result of a target protein to be detected binding to protein detector 30, and a signal monitor 36 that displays the output from signal processing device 34.

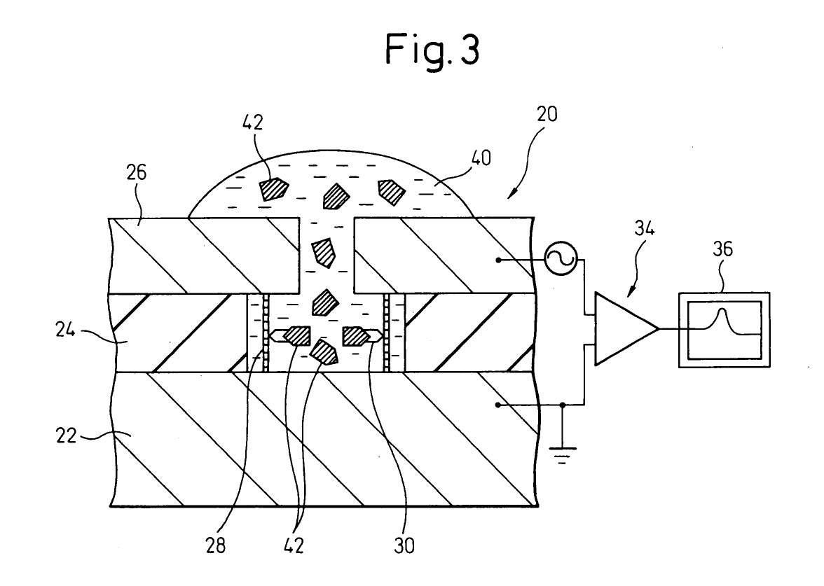

When detecting a protein with protein detection device 20, as shown in FIG. 3, a solution 40 containing a target protein to be detected 42 is supplied to device 20, and solution 40 fills a cavity in which is located DNA 28 to which is attached protein detector 30. A constant voltage or constant current is applied between lower electrode 22 and upper electrode 26 prior to the start of detection work. If the electrical characteristics of DNA 28 have changed as a result of protein 42 having bound to protein detector 30, then that change is detected by signal processing device 34 in the form of a change in the constant current or constant voltage, and then output to monitor 36. As a result, the presence of a target protein to be detected can be detected on a real-time basis.

The quantity of the target protein can also be measured from the magnitude of the electrical signal.

Moreover, if a plurality of protein detection devices having different protein detectors are arranged in the form of an array, the type of protein in a sample can be identified.

In any case, since the protein detection device of the present invention uses electrical signals for detection, it is not necessary to label the target protein to be detected.

### Embodiments

Although the following provides a more detailed explanation of the present invention with reference to its embodiments, the present invention is not limited to these embodiments.

In this embodiment, a protein detection device is explained that uses DNA attached with biotin that specifically binds to avidin protein.

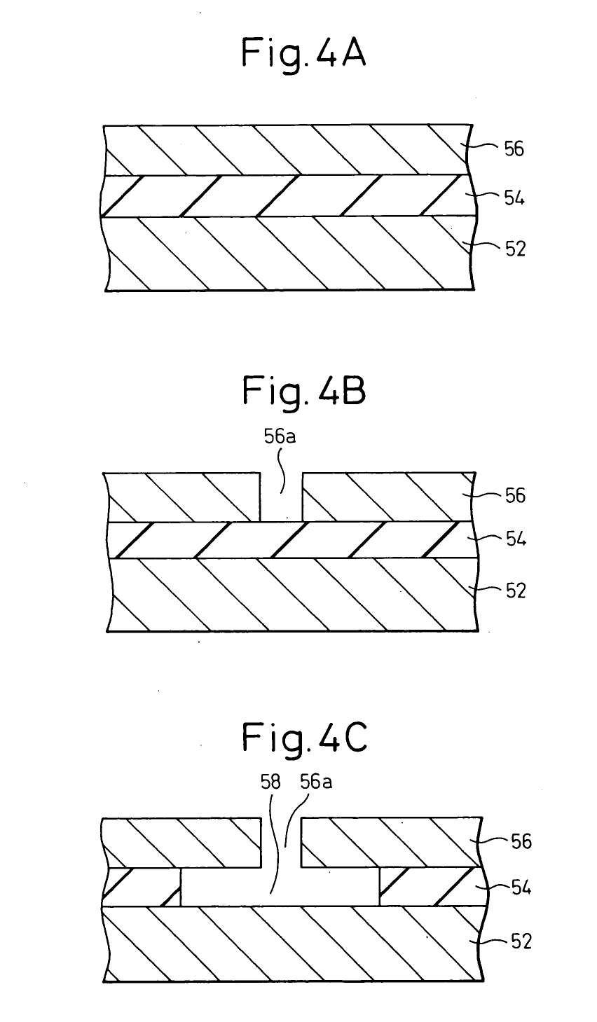

A lower electrode layer 52 made of gold (Au), an insulating layer 54 made of SiO₂, and an upper electrode layer 56 made of gold (Au) are sequentially formed (FIG. 4A) on a silicon substrate (not shown). The thickness of insulating layer 54 is made to be a thickness corresponding to the length of the DNA, for example 10 nanometers, that serves as the bridging member between the opposing electrodes. The thicknesses of lower and upper electrode layers 52 and 56 are made to be, for example, 0.1 micrometers and 0.1 micrometers, respectively. Next, as shown in FIG. 4B, a hole 56a (having a diameter of, for example, 50 micrometers) is formed by, for example, Ar ion etching, in upper electrode layer 56 at the section where the protein detection device is to be fabricated. Subsequently, insulating layer 54 is subjected to under etching by, for example, wet etching to form cavity 58 (having a diameter of, for example, 60 micrometers) below hole 56a as shown in FIG. 4C.

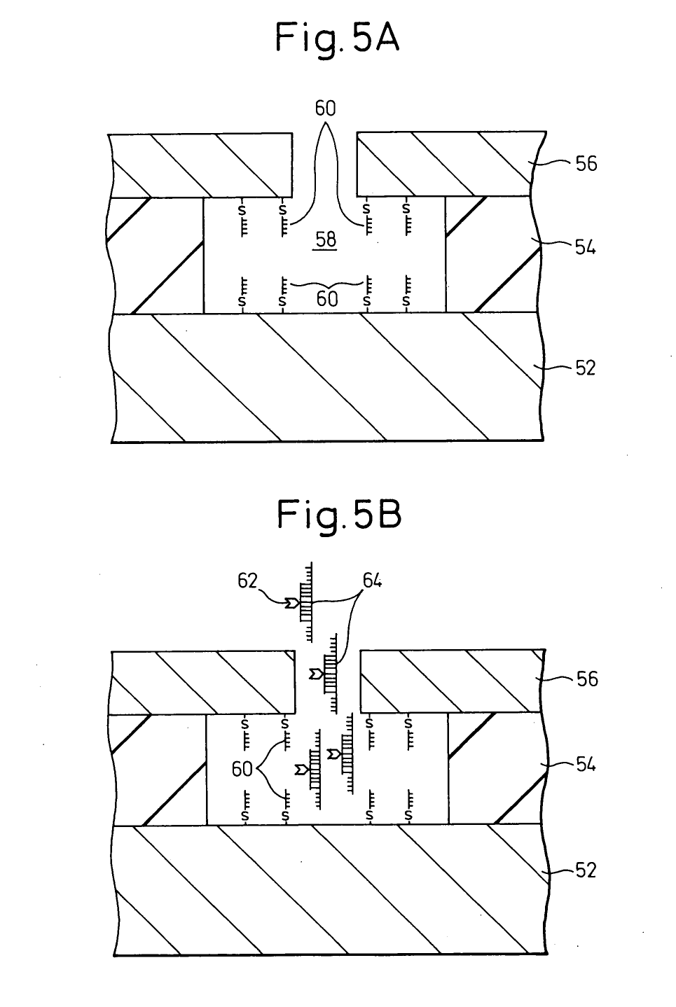

As shown in FIG. 5A, a single-strand DNA 60 having an SH terminal or SS terminal is fixed by self-organization to the exposed surface below upper electrode layer 56 and the exposed surface of lower electrode layer 52, respectively, that demarcate cavity 58. Next, as shown in FIG. 5B, a complementary strand chain DNA 64 is supplied in which biotin 62 is attached to single-strand DNA, and then conjugated with the single-strand DNA 60 fixed to electrode layers 52 and 56 to obtain a protein detection device.

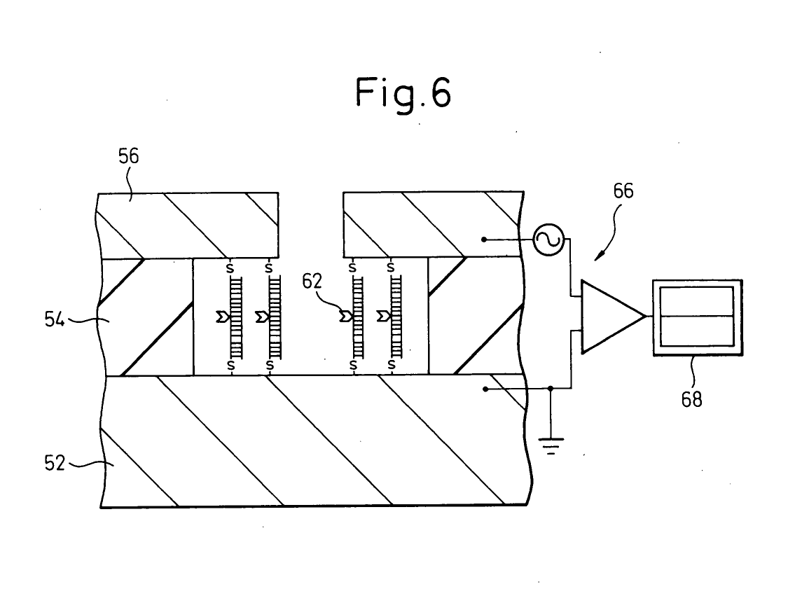

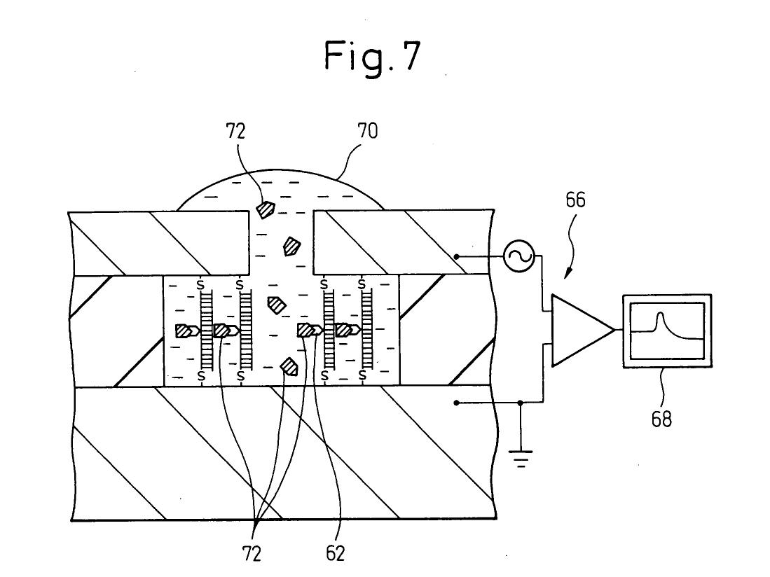

As shown in FIG. 6, lower and upper electrode layers 52 and 56 of the protein detection device are connected to signal processing device 66, and this signal processing device 66 is connected to signal monitor 68. When a voltage is applied between lower and upper electrode layers 52 and 56, and a solution 70 of a test substance is poured into the cavity of the protein detection device (FIG. 7), in the case avidin 72 is present in solution 70, it specifically binds to biotin 62 causing a change in the electrical signal applied between electrodes 52 and 56. The presence and quantity of avidin in the test substance solution can be determined on a real-time basis by carefully monitoring this change.

Although gold is used for the material of the electrode layers in the aforementioned embodiment, a metal material other than gold such as platinum can also be used. In general, the electrode layer material may be any material that is electrically conductive and allows the bridging member to be attached. For example, a semiconductor doped with impurities may also be used as an electrode layer material. The material of the insulating layer may also be any material capable of forming an insulating thin film, and is not limited to the aforementioned SiO₂, but rather may be a material such as SiNₓ. Examples of insulating layer materials other than this type of semiconductor oxide or semiconductor nitride include organic polymer materials such as polyimides and undoped insulating semiconductors.

In the aforementioned embodiment, although an SH or SS terminal of the single-strand DNA is used for fixing the DNA bridging member in the form of a polynucleotide to the electrode layers, the DNA bridging member may also be fixed to the electrode layers by attaching amino groups or carboxyl groups to the electrode surfaces and then attaching the single-strand DNA thereto.

In the aforementioned embodiment, the electrode surfaces other than the sections where the DNA bridging member used for protein detection is bound are left exposed. Electrode surfaces at those sections other than where the bridging member is bound may be protected with an insulator such as a self-assembled membrane (SAM) or they be protected by attaching thereto an insulating organic substance (e.g., epoxy adhesive) or inorganic substance (e.g., metal oxide or semiconductor oxide).

Moreover, although the aforementioned embodiment describes a single protein detection device, by arranging a plurality of devices one-dimensionally or two-dimensionally that contain devices using DNA attached with a protein detector other than biotin for the bridging member, samples containing multiple types of proteins can be tested simultaneously.

### INDUSTRIAL APPLICABILITY

According to the present invention, a sensor and/or device can be provided that is provided with a gap on the nanometer scale formed precisely and with good reproducibility using an extremely inexpensive method. In addition, the presence and quantity of a protein or other target substance to be detected can be detected based on the magnitude of an electrical signal without having to label the target substance to be detected, and the type of target substance to be detected in a sample can be identified by arranging a plurality of devices in the form of an array. This detection and identification can be carried out on a real-time basis. Moreover, since an electrical signal is used to detect a target substance to be detected, in comparison with commonly employed techniques of the prior art involving the observation of fluorescence, there is no need for an elaborate optical device, thereby significantly contributing to reduced size and cost of the device.

1. A cavity electrode structure comprising: a first electrode, an insulating layer located on this first electrode and having a through hole that partially exposes the first electrode, and a second electrode opposed to the exposed surface of the first electrode by protruding towards the inside of the through hole of the insulating layer and provided with an opening that leads to the through hole of the insulating layer; said cavity electrode structure having a cavity that is formed by the exposed surface of the first electrode, the inner walls of the through hole of the insulating layer, and the surface of the second electrode that opposes the first electrode.

2. A cavity electrode structure according to claim 1 wherein, the interval between the first electrode and the second electrode is 100 nanometers or less.

3. A cavity electrode structure according to claim 1 wherein, the width or diameter of the through hole of the insulating layer is 1 to 1000 micrometers.

4. A cavity electrode structure according to claim 1 wherein, the width or diameter of the opening of the second electrode is 1 to 100 micrometers.

5. A cavity electrode structure according to claim 1 wherein, the material of the first electrode and the second electrode is a metal or semiconductor doped with an impurity.

6. A sensor comprising: a cavity electrode structure comprising a first electrode, an insulating layer located on this first electrode and having a through hole that partially exposes the first electrode, and a second electrode opposed to the exposed surface of the first electrode by protruding towards the inside of the through hole of the insulating layer and provided with an opening that leads to the through hole of the insulating layer; said cavity electrode structure having a cavity that is formed by the exposed surface of the first electrode, the inner walls of the through hole of the insulating layer, and the surface of the second electrode that opposes the first electrode; the sensor further comprising an electrically conductive bridging member of which one end is fixed to the exposed surface of the first electrode of said electrode structure, while the other end is fixed to the opposing surface of the second electrode, and which has a site that specifically binds to a target substance to be detected.

7. A sensor according to claim 6 wherein, the interval between the first electrode and the second electrode is 100 nanometers or less.

8. A sensor according to claim 6 wherein, the width or diameter of the through hole of the insulating layer is 1 to 1000 micrometers.

9. A sensor according to claim 6 wherein, the width or diameter of the opening of the second electrode is 1 to 100 micrometers.

10. A sensor according to any of claim 6 wherein, the material of the first electrode and the second electrode is a metal or semiconductor doped with an impurity.

11. A sensor according to any of claim 6 wherein, the bridging member is a high molecular weight biomolecule.

12. A sensor according to claim 11 wherein, the high molecular weight biomolecule is a polynucleotide.

13. A sensor according to claim 11 wherein, the site that specifically binds to a target substance to be detected is composed of an antibody, aptamer or low molecular weight organic substance.

14. A sensor according to claim 11 wherein, the high molecular weight biomolecule is an oligonucleotide having a complementary sequence residue to the target substance to be detected.

15. A protein detection device comprising: a cavity electrode structure comprising a first electrode, an insulating layer located on this first electrode and having a through hole that partially exposes the first electrode, and a second electrode opposed to the exposed surface of the first electrode by protruding towards the inside of the through hole of the insulating layer and provided with an opening that leads to the through hole of the insulating layer; said cavity electrode structure having a cavity that is formed by the exposed surface of the first electrode, the inner walls of the through hole of the insulating layer, and the surface of the second electrode that opposes the first electrode; the protein detection device further comprising an electrically conductive bridging member of which one end is fixed to the exposed surface of the first electrode of said electrode structure, while the other end is fixed to the opposing surface of the second electrode, and which has a site that specifically binds to a target protein to be detected.

16. A protein detection device according to claim 15 wherein, the interval between the first electrode and the second electrode is 100 nanometers or less.

17. A protein detection device according to claim 15 wherein, the width or diameter of the through hole of the insulating layer is 1 to 1000 micrometers.

18. A protein detection device according to claim 15 wherein, the width or diameter of the opening of the second electrode is 1 to 100 micrometers.

19. A protein detection device according to claim 15 wherein, the material of the first electrode and the second electrode is a metal or semiconductor doped with an impurity.

20. A protein detection device according to claim 15 wherein, the bridging member is a high molecular weight biomolecule.

21. A protein detection device according to claim 20 wherein, the high molecular weight biomolecule is a polynucleotide.

22. A protein detection device according to claim 20 wherein, the site that specifically binds to a target protein to be detected is composed of an antibody, aptamer or low molecular weight organic substance.

23. A protein detection device according to claim 15 wherein, the material of the insulating layer is an oxide or nitride of a semiconductor or an organic polymer material.

24. A protein detection device according to claim 15 further comprising a signal processing device for processing signals that indicate a change in electrical characteristics that occurs as a result of a target protein to be detected binding to a site that specifically binds to the target protein to be detected, the signal processing device being connected to the lower electrode and the upper electrode.
